(19) United States
(12) Patent Application Publication
(10) Pub. No.: US 2009/0005320 A1
(43) Pub. Date: Jan. 1, 2009

(54) COMPOSITIONS COMPRISING AMINO ACID BICARBONATE AND METHODS OF USE THEREOF

(76) Inventor: Bruce Kneller, Randolph, MA (US)

Correspondence Address: MORSE, BARNES-BROWN & PENDLETON, P.C.;ATTN: PATENT MANAGER, RESERVOIR PLACE, 1601 TRAPELO ROAD, SUITE 205, WALTHAM, MA, 02451, US

(21) Appl. No.: 12/202,979

(22) Filed: Sep. 2, 2008

(51) Int. Cl. A61K 38/05; A61K 38/06; A61K 31/19

(52) U.S. Cl. 514/18; 514/19; 514/557

(57) ABSTRACT

The invention relates to compositions comprising one or more ionic salts, each of said ionic salts consisting of a bicarbonate anion and a cation selected from the group consisting of an amino acid, an amino acid derivative, a di-peptide and a tri-peptide, and to methods of making and using said compositions.

### BACKGROUND OF THE INVENTION

Nutritional supplements and approaches for enhancing an athlete's muscle performance and the like (e.g., reducing fatigue, increasing strength, increasing endurance, etc.) have become popular exigencies in various sports and bodybuilding regimes. However, as athletes continually strive for improved muscle performance, there is a continuing need for new, more effective technologies to aid in increasing performance.

### SUMMARY OF THE INVENTION

The invention relates to a nutritional supplement or nutraceutical, e.g., a food supplement, a food composition, comprising, consisting essentially of or consisting of an amino acid bicarbonate salt or an amino acid bicarbonate salt derivative, and to methods of making and using said salt. In the methods of the invention one or more amino acid bicarbonate salts or amino acid bicarbonate salt derivatives can be administered alone or in combination with mineral bicarbonate salts such as sodium bicarbonate.

In one embodiment the invention relates to a composition comprising one or more ionic salts, each of said ionic salts consisting of a bicarbonate anion and a cation selected from the group consisting of an amino acid, amino acid derivative, a di-peptide and a tri-peptide. In one embodiment the cation portion of at least one of said ionic salts has a molecular weight of from about 40 to about 1,000, inclusive. As used herein, the term “about” refers to a +/−10% variation from the nominal value. It is to be understood that such a variation is always included in any given value provided herein, whether or not it is specifically referred to.

In another embodiment the cation portion of at least one of said one or more ionic salts consists of from about 50% to about 95% of the total mass of the ionic salt, inclusive. In a further embodiment the amount of at least one of said one or more ionic salts is from about 0.100 grams to about 50.00 grams, inclusive. In specific embodiments the cation is selected from the group consisting of leucine, arginine, ornithine, isoleucine, valine, taurine, glutamine, tyrosine, tryptophan, methionine, glycine, alanine, histidine, phenylalanine, lysine, threonine, asparagine, serine, proline, cysteine, carnitine, carnosine, valenine, and derivatives thereof. In preferred embodiments the composition further comprises a mineral bicarbonate.

The invention also relates to a method of increasing athletic performance in an animal comprising administering one or more ionic salts, each of said ionic salts consisting of a bicarbonate anion and a cation selected from the group consisting of an amino acid, amino acid derivative, a di-peptide and a tri-peptide. In particular embodiments, the method further comprises administering said one or more ionic salt in conjunction with one or more mineral bicarbonate salts. In one embodiment the cation portion of at least one of said one or more ionic salts has a molecular weight of from about 40 to about 1,000, inclusive. In another embodiment the cation portion of at least one of said one or more ionic salts consists of from about 50% to about 95% of the total mass of the ionic salt, inclusive. In a further embodiment the amount of at least one of said one or more ionic salts orally administered is from about 0.100 grams to about 50.00 grams, inclusive.

In preferred embodiments the cation is selected from the group consisting of leucine, arginine, ornithine, isoleucine, valine, taurine, glutamine, tyrosine, tryptophan, methionine, glycine, alanine, histidine, phenylalanine, lysine, threonine, asparagine, serine, proline, cysteine, carnitine, carnosine, valenine, and derivatives thereof.

In specific embodiments said one or more ionic salts are administered orally to said animal in the form of a powder, tablet, capsule, liquid solution, liquid suspension, gel preparation, emulsion, or chewable wafer. The invention also relates to methods wherein said one or more ionic salts increases athletic performance by buffering plasma H+ levels in said animal during, before, and/or after exercise; helps to induce a state of metabolic alkalosis in said animal; and/or helps to increases the skeletal muscle level of phosphocreatine in said animal helps to increases plasma lactate level in said animal.

### DETAILED DESCRIPTION OF THE INVENTION

Sodium bicarbonate (molecular weight of about 84.007, chemical formula=NaHCO₃) is an ionic salt consisting of a sodium (Na+) cation and a bicarbonate (HCO₃—) anion. The oral ingestion of sodium bicarbonate is known to improve athletic performance by increasing intracellular and extracellular H⁺ ion buffering capacity via induction of metabolic alkalosis. Particular improvements in various parameters of athletic performance are discussed at length in the literature (e.g., J Appl Physiol. 101(3):918-25 (September 2006); J Sports Sci. 10(5):425-35 (October 1992); J Sports Med Phys Fitness 41(4):456-62 (December 2001); Med Sci Sports Exerc 38(10):1746-53 (October 2006); Int J Sports Med. Nov. 14, 2007; J Appl Physiol. 99(5):1668-75 (November 2005)).

Oral administration of bicarbonate in the form of sodium bicarbonate improves athletic performance in trained and untrained members of both sexes in a variety of athletic settings. The increase in performance produced by the oral ingestion of NaHCO₃ is scalar and is directly related to the quantity of NaHCO₃ ingested.

However, there exist significant safety issues related to use of NaHCO₃. Typically, the effective oral dosage utilized is about 0.3 grams of NaHCO₃ per kilogram of body mass. Thus, a human with a body mass of 70 kilograms would need to ingest about 21 grams of NaHCO₃ to see a beneficial effect. As about 27.3% of the molecular weight of NaCO₃ consists of sodium (Na+), ingesting 21 grams of NaCO₃ would result in the ingestion of about 5.47 grams of sodium. The United States Recommended Daily Allowance (RDA) of sodium in a healthy, adult male is 2,400 mg (2.40 grams), while the National Academy of Sciences' Institute of Medicine suggests a daily allowance of sodium as low as 1,500 mg (1.50 grams). Thus, the ingestion of 0.3 grams of NaHCO₃ per kilogram of body mass in a 70 kg human would result in the ingestion of 2.23 to 3.64 times the suggested total daily dose of sodium.

Excessive sodium consumption can lead to a physiological condition called hypernatremia, a serious and potentially life-threatening condition. Symptoms of hypernatremia include nausea, vomiting, hypertension, dizziness, renal stress, heart failure, and, if untreated, coma and death. Additionally, it is well known to those in the art that a high plasma sodium level in humans is associated with muscle weakness, fatigue, and an overall decrease in performance. Thus, it is clear that improved agents are needed for delivery of bicarbonate in amounts sufficient to improve athletic performance without providing undesirable amounts of sodium to the recipient.

Various amino acids and their derivatives have been successfully utilized as nutraceutically acceptable supplements that increase athletic performance. These amino acids are often administered orally in the form of ionic salts in which the amino acid portion of the salt is a cation. Examples include, but are not limited to, leucine, isoleucine and valine.

Leucine (C₆H₁₃NO₂, RN: 328-39-2, molecular weight about 131.174), Isoleucine (C₆H₁₃NO₂, RN: 443-79-8, molecular weight about 131.174), and Valine (C₅H₁₁O₂, RN: 516-06-3, molecular weight about 117.147) possess a similar structure with a branch-chain residue and are thus known as branch chain amino acids (BCAA). They are essential amino acids for animals and share a common membrane transport system and enzyme for their transamination and oxidative decarboxylation, indicating that they are closely related in their metabolic fate.

BCAAs account for about 35-40% of the dietary essential amino acids in body protein and about 14-18% of the total amino acids in muscle proteins (J Nutr. 2003; 133:1383-9 and J Nutr. 2004; 134 (Suppl):S968-73). The muscle mass of humans is about 40% of total body weight; the muscle protein pool therefore represents a very large reservoir of BCAA in the body. One the other hand, animals have a free amino acid pool, which appears to be constant, and the content of free BCAA in human skeletal muscle is only about 0.100 gram/kilogram of muscle. This pool of free BCAA is extremely small when compared with the BCAA content of muscle proteins. The concentration of BCAA in human blood (0.3-0.4 mM) is relatively high when compared to that of other amino acids except glutamine (J Clin Invest. 1974; 53:1080-90 and J Clin Invest. 1976; 57:987-99). However, the amount of BCAA in human blood is also very small when compared to that in muscle proteins. Recent studies have demonstrated that free BCAA, especially leucine, play a very important role in protein metabolism; leucine promotes protein synthesis and inhibits protein degradation via mechanisms involving the mammalian target rapamycin (Proc Nutr Soc. 2004; 63:351-6 and J Biol Chem. 2000; 275:29900-6). The findings suggest that leucine is not only a building block of proteins but also a modulator of protein metabolism.

Oral BCAA supplements (77 mg/kg/bodyweight) before exercise have been reported to increase intracellular and artertial BCAA level during exercise, resulting in a suppression of endogenous muscle protein breakdown (Am J Physiol. 1994; 267:E1010-22). Oral BCAA administration (12 g/day for 2 weeks and additionally 20 g each before and after exercise test) also reportedly suppresses the rise of plasma creatine kinase activity for several days after exercise (J Sports Med Phys Fitness. 2000; 40:240-6).

Glutamine (C₅H₁₀N₂O₃, RN: 56-85-9, molecular weight about 146.145) is another amino acid that has been demonstrated to potentially possess an athletic performance enhancing effect when orally administered. Taurine (C₂H₇NO₃S, RN: 107-35-7, molecular weight about 125.147) is yet another amino acid that has been demonstrated to potentially possess an athletic performance enhancing effect when orally administered. Intense exercise increases oxidative stress and thus, damage muscle tissue. Taurine is present in high concentration in skeletal muscle and may play a role in cellular defenses against free-radical mediated damage. Tyrosine (C₉H₁₁NO₃, RN: 556-03-6, molecular weight about 181.19) is yet another amino acid that has been demonstrated to potentially possess an athletic performance enhancing effect when orally administered.

It is clear by the above examples that amino acids have been demonstrated to induce performance enhancing effects when administered to animals. While leucine, isoleucine, valine, taurine, glutamine, and tyrosine are specifically discussed herein, the invention should not be construed to be limited to these amino acids or derivatives of these amino acids. Other amino acids and their derivatives, such as tryptophan, methionine, glycine, alanine, histidine, phenylalanine, lysine, arginine, ornithine, threonine, asparagine, serine, proline and cysteine, recognized by those skilled in the art as having a potential performance enhancing effect when administered orally, can readily serve as cations in stable, ionic salts with bicarbonate anions. Specifically, any amino acid or amino acid derivative or small di- or tri-peptide with a molecular weight from about 40 to about 1,000 can serve as an appropriate cation to form an ionic salt with a bicarbonate anion in accordance with the invention.

Additionally, this invention includes the use of multiple amino acid or amino acid derivative ionic bicarbonate salts together. As non-limiting examples, the oral administration of leucine bicarbonate, isoleucine bicarbonate and valine bicarbonate (three bicarbonate salts) is encompassed within the invention, as well as administration of glutamine bicarbonate and taurine bicarbonate. Those skilled in the art will recognize that the administration of more than one amino acid bicarbonate or amino acid bicarbonate derivative or the ingestion of one or more amino acid bicarbonates or amino acid bicarbonate derivatives with one or more mineral bicarbonate salts such as sodium bicarbonate need not be concomitant as long as administration is proximate in time. For example, the individual bicarbonate salts may be administered within 24 hours of each other.

Further, this invention includes the use of one or more amino acid or amino acid derivative ionic bicarbonate salts used in conjunction with one or more mineral bicarbonate salts, such as, but not limited to, sodium bicarbonate. For example, the oral administration of leucine bicarbonate with sodium bicarbonate dramatically reduces the amount of sodium bicarbonate needed to produce a buffering effect of H+ in the body, leading to an increase in athletic performance while substantially reducing the amount of exogenous sodium ingested. This would also simultaneously allow for the ergogenic benefit of leucine. The addition of tyrosine bicarbonate to the above example—the ingestion of leucine bicarbonate, tyrosine bicarbonate, and sodium bicarbonate—would further decrease the amount of orally ingested sodium bicarbonate required to induce an increase in athletic performance based on H+ buffering, while simultaneously providing the ergogenic benefit of additional tyrosine and leucine.

The compositions of the invention can beneficially be formulated as a nutritional supplement. As used herein, a nutritional supplement, also known as food supplement or dietary supplement, is a preparation for supplying effective agents that are missing or are not consumed in sufficient quantity in an individual's diet to have a desired effect. Typically the nutritional supplement is orally ingested, but it may also be administered via other routes.

Amino acid bicarbonates can be made by any suitable method; such methods will be readily apparent to the skilled artisan.

In some embodiments of the invention the composition (i.e., amino acid bicarbonate or a nutritional supplement comprising amino acid bicarbonate) is formulated as a tablet, capsule, caplet, powder, suspension, gel preparation, aqueous solution, solid food form, or liquid dosage form such as elixirs, syrups, dispersed powders, granules or emulsions. In one embodiment the composition is particularly formulated for oral use.

In addition, the amino acid bicarbonate can be administered before, concurrent with or after other optional components such as other active ingredients. In some embodiments the nutritional supplement composition comprising amino acid bicarbonate contains one or more of the following ingredients, preferably as an active ingredient:
Carbohydrates including, but not limited to, isomaltulose, trehalose, maltodextrin, glucose, sucrose, fructose, lactose, amylose, and/or ribose;
Water soluble vitamins including, but not limited to, Vitamin C, Vitamin B1, Vitamin B2, Vitamin B3, Vitamin B6, Vitamin B12, and/or Vitamin K (and derivatives);
Minerals including, but not limited to, calcium, sodium, potassium, chromium, vanadium, magnesium, and/or iron (and derivatives) (preferably in amounts less than the RDA);
Amino acids including, but not limited to, L-arginine, L-ornithine, L-glutamine, L-tyrosine, L-taurine, L-leucine, L-isoleucine, and/or L-valine (and derivatives);
Nutraceutically acceptable stimulants including, but not limited to, methylxanthines (e.g.—caffeine) and/or glucuronolactone (and derivatives);
Nutraceutically acceptable hypoglycemic agents including, but not limited to, alpha-lipoic acid and its derivatives, cinnamon bark, bitter melon extracts, Gymnema Sylvestre extracts, 4-hydroxy-isoleucine, corosolic acid, and/or D-pinitol (and derivatives);
Creatine and its salts (e.g., creatine monohydrate), esters (e.g., creatine ethyl ester), chelates, amides, ethers (and derivatives);
Adenosine triphosphates and its disodium salt;
Glycerol and glycerol monostearate;
Mannitol;
Sorbitol; and

Dextran.

Preferably the composition comprises from about 5% to about 100% amino acid bicarbonate, more preferably about 20% to about 100% amino acid bicarbonate, and even more preferably about 50% to about 100% amino acid bicarbonate.

As used herein, the terms “nutraceutical” and nutraceutically acceptable” are used herein to refer to any substance that is a food or part of a food and provides medical or health benefits, including the prevention and treatment of disease. Hence, compositions falling under the label “nutraceutical” or “nutraceutically acceptable” may range from isolated nutrients, nutritional or dietary supplements, and specific diets to genetically engineered designer foods, herbal products, and processed foods such as cereals, soups, and beverages. In a more technical sense, the term has been used to refer to a product isolated or purified from foods, and generally sold in medicinal forms not usually associated with foods and demonstrated to have a physiological benefit or provide protection against chronic disease.

As used herein, the term “derivative” can include salts, esters and/or ethers (e.g., any carboxylic acid ester or ether in which the ester or ether contains between 2 and 22 carbon atoms), chelates (a complex with a metal ion including but not limited to iron, zinc, manganese, magnesium, copper, calcium and mixtures thereof; see, e.g., U.S. Pat. Nos. 4,020,158; 4,167,564; 4,216,143; 4,216,144; 4,599,152; and 4,774,089, incorporated by reference herein), lactone forms, hydrates, or complexes of stated chemicals. As used herein, a pharmacologically acceptable salt is an ionic salt with an acid as an anion which does not give rise to any unwanted side effects or toxic effects. Such acids are known to those skilled in the art. Non-limiting examples of such salts are: chloride; bromide; iodide; aspartate; citrate; tartrate; phosphate; fumarate; glycerophosphate; glucose phosphate; lactate; maleate; mucate; orotate; oxalate; suphate; trichloroacetate; trifluoroacetate; methane sulphonate; acetate; succinate; and gluconate. A list of FDA-approved pharmacologically acceptable acids can be found in Int. J. Pharm. 33:201-217 (1986), incorporated herein by reference in its entirety.

Such derivatives can also include stereoisomers or structural isomers, so long as the derivative operates similarly and produces the desired effect. Alternatively, the derivative can be a precursor to the stated chemical, which subsequently undergoes a reaction in vivo to yield the stated chemical or derivative thereof. By way of non-limiting example only, ubiquinol is a useful derivative of ubiquinone, and acetyl-L-carnitine, carnitine hydrochloride, and the D and L stereoisomers of carnitine are useful derivatives of L-carnitine.

The compositions may contain pharmaceutically, e.g., nutraceutically, acceptable excipients, according to methods and procedures well known in the art. As used herein, “excipient” refers to substances that are typically of little or no therapeutic value, but are useful in the manufacture and compounding of various pharmaceutical preparations and which generally form the medium of the composition. These substances include, but are not limited to, coloring, flavoring, and diluting agents; emulsifying, dispersing and suspending agents, ointments, bases, pharmaceutical solvents; antioxidants and preservatives; and miscellaneous agents. Suitable excipients are described, for example, in Remington's Pharmaceutical Sciences, which is incorporated herein by reference in its entirety.

The nutritional supplement compositions according to the present invention can further comprise one or more acceptable carriers. A wide number of acceptable carriers are known in the nutritional supplement arts, and the carrier can be any suitable carrier. The carrier need only be suitable for administration to animals, including humans, and be able to act as a carrier without substantially affecting the desired activity of the composition. Also, the carrier(s) may be selected based upon the desired administration route and dosage form of the composition. For example, the nutritional supplement compositions according to the present invention are suitable for use in a variety of dosage forms, such as liquid form and solid form. In desirable embodiments, as discussed below, the nutritional supplement compositions comprise a solid dosage form, such as a tablet or capsule. Examples of suitable carriers for use in tablet and capsule compositions include, but are not limited to, organic and inorganic inert carrier materials such as gelatin, starch, magnesium stearate, talc, gums, silicon dioxide, stearic acid, cellulose, and the like. Desirably, the carrier is substantially inert, but it should be noted that the nutritional supplement compositions of the present invention may contain further active ingredients in addition to amino acid bicarbonate.

### Methods of Use

The compositions and methods of the present invention may provide significant increase or improvement in athletic performance, e.g., muscle size, and/or muscle strength, and/or muscle endurance in individuals. As used herein, “athletic performance” and/or “athletic functions” refers to the sum of physical attributes which can be dependent to any degree on skeletal muscle contraction. For example, athletic performance and/or athletic functions include, but are not limited to, maximal muscle power, muscular endurance, running speed and endurance, swimming speed and endurance, throwing power, lifting and pulling power.

While it is expected that the compositions and methods of the present invention will be of particular importance to bodybuilders and other athletes, the usefulness of compositions and methods of the invention is not limited to those groups. Rather, any individual may beneficially use the compositions and methods of the invention. Indeed, the disclosed compositions and methods have application to all animals, including mammals, birds and reptiles. As used herein, the term “animal” includes all members of the animal kingdom, preferably mammals (e.g., dogs, horses, cows, mules), more preferably humans. For example, the nutritional supplements of the invention may have beneficial effect for competitive animals (e.g., racehorses, show horses, racing dogs (e.g., greyhounds), bird dogs, show dogs) and work animals (e.g., horses, mules and the like) in whom an increase in muscle performance is desirable.

The compositions according to the present invention may be employed in methods for supplementing the diet of an individual, e.g., an athlete, and/or for enhancing an individual's muscle mass and/or muscle size and/or strength, and/or endurance. Accordingly, the present invention provides methods of supplementing the dietary intake of an individual comprising administering to the individual an effective amount of a composition (e.g., amino acid bicarbonate or a nutritional supplement comprising amino acid bicarbonate) according to the present invention to increase athletic performance or athletic function is said individual. The invention also relates to methods of improving athletic performance and/or athletic function in an individual comprising administering an effective amount of amino acid bicarbonate (alone or in combination with other agents, e.g., in a nutritional supplement) to the individual.

As used herein, an “effective amount” of compositions of the present invention is defined as an amount effective, at dosages and for periods of time necessary, to achieve the desired result. The effective amount of compositions of the invention may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present invention may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

The embodiments set forth in the present application are provided only to illustrate various aspects of the invention and additional embodiments and advantages of the food supplements and methods of the present invention will be apparent to those skilled in the art. The teachings of all references cited herein are incorporated herein by reference in their entirety.

### EXAMPLES

The servings set forth in these examples are designed for a 2,500 calorie daily diet. Daily values can be increased or decreased depending on the needs of the individual athlete.

### Example 1

In this example, an athlete consumes two servings of the composition as described herein daily: a serving of the food supplement six hours before and then 1 hour before his/her workout. Each serving of the food supplement is about 30.00 grams and contains the following:

L-Leucine Bicarbonate 30.00 grams.

Each approximate 30.00 gram serving is administered mixed in about 32 ounces of cold water to provide a liquid drink.

### Example 2

In this example, an athlete consumes one serving of the food supplement as described herein ad libitum, preferably approximately 90 minutes before a workout. Each serving of the food supplement is about 28 grams and comprises:

Sodium Bicarbonate 10.00 grams; L-Glutamine

Bicarbonate 10.00 grams; L-Taurine Bicarbonate 5.00 grams; and L-Tyrosine Bicarbonate 3.00 grams.

Each approximate 28.00 gram serving is administered mixed in about 32 ounces of cold water to provide a liquid drink.

### Example 3

In this example, an athlete consumes two servings of the food supplement as described herein daily: a serving of the food supplement two hours before and then two hours before his/her workout. Each serving of the food supplement is about 40.003 grams and contains the following:

L-Leucine Bicarbonate 3.00 grams; L-Valine Bicarbonate 1.50 grams; L-Isoleucine Bicarbonate 1.50 grams; L-Glutamine Bicarbonate 4.00 grams; β-Alanine Bicarbonate 4.00 grams; Coenzyme Q-10 0.200 grams; Disodium Adenosine TriPhosphate 0.200 grams; Glucuronolactone 0.600 grams; Corosolic Acid 0.003 grams; Maltodextrin 10.00 grams; Isomaltulose 10.00 grams; and Ribose 5.00 grams.

Each approximate 40.003 gram serving is administered mixed in about 24 ounces of cold water to provide a liquid drink. An additional 16 ounces of cold water may be consumed after the food supplement liquid drink is consumed.

What is claimed is:

1. A method of increasing athletic performance in an animal comprising administering one or more ionic salts, each of said ionic salts consisting of a bicarbonate anion and a cation selected from the group consisting of an amino acid, an amino acid derivative, a di-peptide and a tri-peptide.

2. A method according to claim 1, further comprising administering said one or more ionic salt in conjunction with one or more mineral bicarbonate salts.

3. A method according to claim 1 wherein the cation portion of at least one of said one or more ionic salts has a molecular weight of from about 40 to about 1,000, inclusive.

4. A method according to claim 1 wherein the cation portion of at least one of said one or more ionic salts consists of from about 50% to about 95% of the total mass of the ionic salt, inclusive.

5. A method according to claim 1 wherein the amount of at least one of said one or more ionic salts orally administered is from about 0.100 grams to about 50.00 grams, inclusive.

6. A method according to claim 1 wherein the cation is selected from the group consisting of leucine, arginine, ornithine, isoleucine, valine, taurine, glutamine, tyrosine, tryptophan, methionine, glycine, alanine, histidine, phenylalanine, lysine, threonine, asparagine, serine, praline, cysteine, carnitine, carnosine, valenine, and derivatives thereof.

7. A method according to claim 1 wherein said one or more ionic salts are administered orally to said animal in the form of a powder, tablet, capsule, liquid solution, liquid suspension, gel preparation, emulsion, chewable bar or chewable wafer.

8. A method according to claim 1 wherein said one or more ionic salts increases athletic performance by buffering plasma H+ levels in said animal during, before, and/or after exercise.

9. A method according to claim 1 wherein said one or more ionic salts helps to induce a state of metabolic alkalosis in said animal.

10. A method according to claim 1 wherein said one or more ionic salts helps to increases the skeletal muscle level of phosphocreatine in said animal.

11. A method according to claim 1 wherein said one or more ionic salts helps to increases plasma lactate level in said animal.

12. A method according to claim 2 wherein the cation portion of at least one of said ionic salts has a molecular weight of from about 40 to about 1,000, inclusive.

13. A method according to claim 2 wherein the cation portion of at least one of said one or more ionic salts consists of from about 50% to about 95% of the total mass of the ionic salt, inclusive.

14. A method according to claim 2 wherein the amount of at least one of said one or more ionic salts orally administered is from about 0.100 grams to about 50.00 grams, inclusive.

15. A method according to claim 2 wherein said cation is selected from the group consisting of leucine, arginine, ornithine, isoleucine, valine, taurine, glutamine, tyrosine, tryptophan, methionine, glycine, alanine, histidine, phenylalanine, lysine, threonine, asparagine, serine, praline, cysteine, carnitine, carnosine, valenine, and derivatives thereof.

16. A method according to claim 2 wherein said one or more ionic salts are administered orally to said animal in the form of a powder, tablet, capsule, liquid solution, liquid suspension, gel preparation, emulsion, chewable bar or chewable wafer.

17. A method according to claim 2 wherein said one or more ionic salts increases athletic performance by buffering plasma H+ levels in said animal during, before, and/or after exercise.

18. A method according to claim 2 wherein said one or more ionic salts helps to induce a state of metabolic alkalosis in said animal.

19. A method according to claim 2 wherein said one or more ionic salts helps to increases the skeletal muscle level of phosphocreatine in said animal.

20. A method according to claim 2 wherein said one or more ionic salts helps to increases plasma lactate level in said animal.

21. A composition comprising one or more ionic salts, each of said ionic salts consisting of a bicarbonate anion and a cation selected from the group consisting of an amino acid, amino acid derivative, a di-peptide and a tri-peptide.

22. A composition according to claim 21 wherein the cation portion of at least one of said ionic salts has a molecular weight of from about 40 to about 1,000, inclusive.

23. A composition according to claim 21 wherein the cation portion of at least one of said one or more ionic salts consists of from about 50% to about 95% of the total mass of the ionic salt, inclusive.

24. A composition according to claim 21 wherein the amount of at least one of said one or more ionic salts is from about 0.100 grams to about 50.00 grams, inclusive.

25. A composition according to claim 21 wherein said cation is selected from the group consisting of leucine, arginine, ornithine, isoleucine, valine, taurine, glutamine, tyrosine, tryptophan, methionine, glycine, alanine, histidine, phenylalanine, lysine, threonine, asparagine, serine, praline, cysteine, carnitine, carnosine, valenine, and derivatives thereof.

26. A composition according to claim 21 which is a nutritional supplement.
